(19) United States
(12) Patent Application Publication
(10) Pub. No.: US 2011/0217262 A1
(43) Pub. Date: Sep. 8, 2011

(54) Treatment of Ocular Surface Disorders by Increasing Conjunctival Vascular Permeability

(76) Inventors: Julia A. Kornfield, Pasadena, CA (US); Robert H. Grubbs, South Pasadena, CA (US); Choon Woo Lee, Pasadana, CA (US); Daniel Schwartz, San Francisco, CA (US); Phoebe Lin, Durham, NC (US); Keith Duncan, San Francisco, CA (US)

(21) Appl. No.: 13/042,437

(22) Filed: Mar. 7, 2011

Related U.S. Application Data
(60) Provisional application No. 61/311,235, filed on Mar. 5, 2010.
(60) Provisional application No. 61/312,780, filed on Mar. 11, 2010.

(51) Int. Cl. A61K 38/20; A61K 31/522; A61K 31/417; A61K 31/4045; A61K 38/18; A61K 38/02; A61K 31/685; A61K 38/19; A61K 31/7076; A61K 31/265; A61K 31/235; C12Q 1/02; C07C 69/96; A61K 31/27; A61P 27/02

(52) U.S. Cl. 424/85.2; 514/252.16; 514/399; 514/415; 514/8.1; 514/20.8; 514/77; 424/85.1; 514/47; 514/512; 514/534; 435/29; 558/273; 514/490

(57) ABSTRACT

A method of treating an ocular surface disorder in a subject in need of such treatment is provided. The method includes exposing conjunctival tissue of the subject to an effective amount of a vasopermeability agent that increases conjunctival vascular permeability. In some embodiments, the agent is a nitric oxide donor, which may be in a sustained release form. A method of screening a substance for treating an ocular surface disorder is also provided.

### CROSS-REFERENCE TO RELATED APPLICATIONS

This application claims the benefit of Provisional Patent Application No. 61/311,235, filed on Mar. 5, 2010, and Provisional Patent Application No. 61/312,780, filed on Mar. 11, 2010, all of which are incorporated by reference herein.

### BACKGROUND

1. Field of the Invention

The invention relates to methods and compositions for treating surface disorders of the eye.

2. Related Art

Dry eye is a highly prevalent, visually debilitating group of conditions previously labeled as dry eye syndrome or dysfunctional tear syndrome. More recently, the term dry eye disease (DED) has been established as the accepted term in a published report by the International Dry Eye Workshop (DEWS) (Behrens et al, Cornea 2006). DED comprises various conditions that manifest with symptoms of ocular irritation and blurred vision associated with decreased tear production and rapid evaporation of the tear film. Many patients also describe ocular fatigue, which seems to be the result of the work of increased blinking as a compensatory mechanism for a rapidly evaporating tear film. DED is increasingly prevalent with age, affecting 5% of the adult population during the fourth decade and 10-15% of those over the age of 65. It is estimated that among Americans 50 years or older, just less than 5 million are affected with moderate to severe dry eye disease (Schaumberg, Am J Ophthalmol 2003). Risk factors for DED include increased age, female gender, hormonal changes (primarily a reduction in androgens), systemic autoimmune disease, decreased corneal sensation, refractive surgery, blinking abnormalities, drug toxicity, viral infections (such as HIV), and vitamin A deficiency.

DED is often difficult to treat, with standard therapies such as frequent artificial tears often ineffective in treating the more advanced cases likely because they do not provide the epitheliotrophic and adhesive factors found in the normal tear film. Since it was first described in 1984 by Fox et al, autologous serum tears have become a potentially effective approach in treating ocular surface disorders. Autologous serum tears are preparations of a patient's own serum formulated for use as eye drops. Tsubota et al reported a case series of keratoconjunctivitis sicca patients with Sjogren's syndrome in which symptoms and rose bengal staining decreased significantly after four weeks of treatment with 20% autologous serum eye drops applied 6-10 times daily (Tsubota et al 1999). Since then, a number of studies have looked at the use of autologous serum tears in the treatment of DED. In a small case series by Poon et al, all three eyes treated with 100% serum had objective and subjective improvement in severe dry eye manifestations, while 50% serum was effective in 3 out of eight cases (Poon et al, 2001). Schulze et al reported the results of a prospective, randomized, masked clinical trial comparing autologous serum against hyaluronic acid for the treatment of epithelial corneal lesions in diabetic patients undergoing vitrectomy. Epithelial healing time was 4.3 days in the autologous serum group versus 7.1 days in the hyaluronic acid group (Schulze et al 2006).

The mechanism of autologous serum tears providing relief and promoting healing is purportedly related to the presence of growth factors, adhesion factors, and antimicrobial factors found in the serum. For instance, fibronectin, which enhances proliferation of corneal epithelial cells is found at almost 100 times the concentration in serum compared to tears in opened eyes. Vitamin A supports epithelial differentiation and is also found at much higher concentrations in serum than in tears. Epithelial growth factor, which also promotes epithelial proliferation, is present at approximately the same concentration in serum and normal tears (Yamada et al 2008). Albumin, a highly stable protein found at a much higher concentration in serum than in tears, has recently been found to inhibit apoptosis of human corneal and conjunctival cells lines as well as promote healing of corneal erosions in a rat model (Higuchi et al 2007).

### SUMMARY

The inventors have discovered a way of treating ocular surface disorders by exposing the eye to serum components without the need to prepare autologous serum tears. Thus, in one aspect, a method of treating an ocular surface disorder in a subject in need of such treatment is provided. The method includes increasing the vascular permeability of conjunctival blood vessels of the subject, which enables the vessels to leak serum components onto the ocular surface. In some embodiments, the increase in conjunctival vascular permeability occurs by exposing conjunctival tissue of the subject to an effective amount of a vasopermeability agent that increases conjunctival vascular permeability. In some embodiments, including embodiments involving a vasopermeability agent, the increase in conjunctival vascular permeability occurs with minimal or no inflammation of the external eye. The subject may be a person or an animal.

In another aspect, a method of screening a substance for treating an ocular surface disorder is provided. The method includes exposing conjunctival tissue to a substance, and determining if exposure to the substance increases conjunctival vascular permeability with minimal or no inflammation.

### BRIEF DESCRIPTION OF THE DRAWINGS

For a more complete understanding of the present invention, reference is now made to the following descriptions taken in conjunction with the accompanying drawings.

FIG. 1 is a diagram of the chemical structure of nitric oxide donor C-NOD.

FIG. 2 is a panel of photographs of C-NOD injected eyes visualized after 24 hours.

FIG. 3 is a panel of photographs of C-NOD injected eyes visualized after 48 hours.

FIG. 4 is a panel of photographs of C-NOD injected eyes visualized after 72 hours.

FIG. 5 is a panel of graphs showing Evans blue dye in tears following injection of C-NOD, where: ♦, rabbit 1; ▪, rabbit 2; Δ, rabbit 3; X, rabbit 4; *, rabbit 5. FIG. 5A: Evans Blue dye (EBD) in C-NOD injected eye relative to fellow, vehicle (DMSO) injected eye. FIG. 5B: Evans blue dye, as measured by absorbance at 620 nm, in eyes injected with C-NOD.

FIG. 6 is a panel of histological sections of rabbit tissue after C-NOD injection. Eosin and hematoxylin stained sections of rabbit eyes injected with (A) 50 mg/cc C-NOD, (B) 25 mg/cc C-NOD, and (C) 10 mg/cc C-NOD.

FIG. 7 is a panel of graphs of the ratio of albumin to total protein in tears sampled at 1 hour and 5 days post-injection.

FIG. 8 is a panel of graphs of total protein in tears of rabbits injected with C-NOD. Protein amounts are expressed relative to uninjected control eyes.

FIG. 9 is a diagram of the chemical structure of nitric oxide donor B-NOD.

FIG. 10 is a graph of the relative amounts of Evans blue dye in uninjected eyes, eyes injected with B-NOD, and eyes injected with vehicle.

### DETAILED DESCRIPTION

Clinical studies have shown that autologous serum tears can be helpful in the treatment of ocular surface disorders. For example, one study showed that mean epithelialization time was reduced in a group of corneal abrasion patients treated with autologous serum compared to a control group treated with hyaluronic acid (artificial tears) (Schulze, S. D. et al.). Another study showed that patients with persistent corneal epithelial defects that were not sufficiently responsive to conventional therapy were healed upon treatment with autologous serum (Jeng, B. H., et al., Cornea 28:1104-1108, 2009). Despite the efficacy of autologous serum tears in treating ocular surface disease, limitations of this therapy include difficulty in preparation (sterile blood draw, centrifugation, sterile dilution and storage), difficulty in preservation of protein components between dosing (preventing exposure to light and keeping refrigerated), and potential for bacterial contamination. For example, Leite et al. sampled eleven bottles of serum tears after 30 days of use, and six had single or multi-bacterial contamination. As described herein, the inventors have discovered a way of exposing the eye to serum components without the need for topical administration of serum or its components.

In some embodiments, the vascular permeability of conjunctival blood vessels is increased as a way to treat ocular surface disorders.

An ocular surface disorder is an eye disorder or defect such as, but not limited to, dry eye disease, neurotrophic keratopathy, a non-healing ocular epithelial defect, an eye disorder due to Stevens-Johnson syndrome, an eye disorder due to graft versus host disease, Sjogren's syndrome, superior limbic keratoconjunctivitis, ocular cicatricial pemphigoid, recurrent or persistent corneal erosion, or dry eye following corneal refractive procedures (LASIK).

The conjunctiva is a membrane that covers the sclera and that lines the inner surfaces of the eyelid. Conjunctival tissue refers to the entire conjunctiva or to a part of the conjunctiva, such as the part covering the sclera or the part lining the inner eyelid surfaces.

In some embodiments, conjunctival tissue is exposed to a vasopermeability agent. The vasopermeability agent may be a single chemical compound, or may comprise two or more chemical compounds. In addition, the agent may act directly on conjunctival blood vessels to increase conjunctival vascular permeability, or may act indirectly by increasing production of a molecule or molecules that in turn increase conjunctival vascular permeability.

Candidates for agents that increase conjunctival vascular permeability include substances that increase blood vessel vasopermeability generally, such as but not limited to, nitric oxide donors, sildenafil, histamine, serotonin, vascular endothelial growth factor (VEGF), substance P, bradykinin, platelet activating factor (PAF), TNFα, leukotrienes, prostaglandins, interleukins, 5-adenosine diphosphate (ADP), or hyperosmolar agents such as mannitol, or a combination thereof.

Conjunctival vascular permeability activity can be determined by measuring the leakage of serum, serum proteins or other serum components from blood vessels of the conjunctiva following exposure to a substance. For example, a candidate substance can be injected subconjunctivally into a test eye of a subject and leakage of serum albumin from conjunctival blood vessels can be measured using an ELISA assay specific for serum albumin, or total protein can be measured using a colorimetric assay such as a Lowry assay. An increase in serum leakage from the test eye compared to serum leakage from the subject's other (control) eye indicates an increase in conjunctival vascular permeability. Alternatively, a vital dye, such as Evans Blue, can be injected intravenously and leakage of the dye onto the surface of the eye can be used as a measure of conjunctival vascular permeability.

Examples of agents that increase conjunctival vascular permeability are the nitric oxide donors

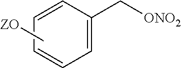

where Z is a functional aryl or alky group,

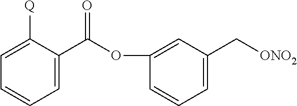

wherein Q is carbamate, carbonate, urea, polymer hydroxyl, or sulfide thiourea,

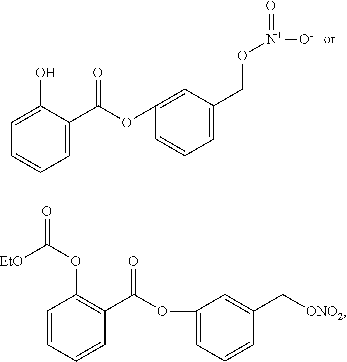

or pharmaceutically acceptable salts thereof, or a combination thereof.

A nitric oxide donor (NOD) is a substance that releases nitric oxide spontaneously or undergoes metabolism to generate nitric oxide. The form of nitric oxide produced can be NO, NO⁻ (nitroxyl anion), NO· (nitric oxide radical), NO⁺ (nitrosonium), or any combination thereof. Nitric oxide donors that have been used as cardiovascular agents include sodium nitroprusside, sodium trioxadimitrate, diazeniumdiolate (NONOate) compounds, S-nitrosothiols, nitroglycerin, amyl nitrate, isosorbide dinitrate, and nicorandil. Not all nitric oxide donors will increase the vascular permeability of conjunctival blood vessel. Nitric oxide donors that are candidate conjunctival vasopermeability agents include, but are not limited to, glyceryl trinitrate, isosorbide mononitrate, pentaerythrityl tetranitrate, S-nitrosothiol, bifunctional nitric oxide donors such as NO-NSAIDs, diazeniumdiolates, or zeolites, or a combination thereof.

In some embodiments, downstream effects of nitric oxide may be exploited to increase conjunctival vascular permeability. For example, nitric oxide can activate guanylate cyclase to catalyze the conversion of guanosine triphosphate to cyclic guanosine monophosphate (cGMP). Inhibition of cGMP phosphodiesterase prevents cGMP breakdown. Because sildenafil inhibits type 5 phosphodiesterase (PDE5), exposure of tissue to sildenafil may result in vascular dilation and permeability alterations similar to those following nitric oxide administration.

A nitric oxide donor or other conjunctival vasopermeability agent may be injected into the subconjunctival space to provide focal nitric oxide release and vascular permeability enhancement. Such an injection beneath the bulbar conjunctiva could be delivered under the upper or lower lid to hide the injection site from view. Alternatively, a topical formulation containing the nitric oxide donor could be applied as an eyedrop to mitigate ocular surface disorders. The rate of release can be controlled by the selection of the substituents on the NO precursor. As demonstrated in the examples, the time of action can be controlled by functionalizing the phenol group with a carbonate that results in slower hydrolysis. Standard chemical principles can be used to produce functional groups that control the activation of the nitric oxide precursor.

Compound B-NOD can be prepared as described in Bing, R. J., et al., The pharmacology of a new nitric oxide donor: B-NOD, Biochem. Biophys. Res. Comm. (2000) 275, 350-353. Compound C-NOD can be prepared by phenol functionalization of B-NOD.

In some embodiments, an effective amount of a conjunctival vascular permeability agent is that amount sufficient to increase leakage of serum, serum components, or serum proteins from conjunctival blood vessels. In some embodiments, an effective amount is that amount of conjunctival vascular permeability agent sufficient to ameliorate, reduce, minimize or limit the extent of an ocular surface disorder or its symptoms.

An increase in vascular permeability may occur with minimal or no external ocular inflammation, or with minimal or no additional external ocular inflammation over that present in a subject's eye prior to treatment. The degree of external ocular inflammation can be determined by a subject's symptoms such as the pain, irritation, foreign body sensation, itching, swelling, or discharge, or a combination thereof, associated with the subject's eye. Clinical signs of external ocular inflammation include conjunctival injection, chemosis, exudation, epiphora, or lid swelling, or a combination thereof. In some embodiments, for example, nitric oxide donor dosing may be adjusted to reduce the signs and symptoms of an ocular surface disorder, such as dry eye, with minimal external ocular inflammation, i.e., with clinically acceptable levels of external ocular inflammation. Clinically acceptable levels are typically determined by a subject's symptoms and/or the external appearance of the eye. For example, minimal inflammation is present when the subject's eye appears normal and the subject is not symptomatic (i.e., no pain, irritation, foreign body sensation, itching, swelling, or discharge, or a combination thereof, is presented by the subject's eye).

Conjunctival tissue may be exposed to a vascular permeability agent by applying the agent topically or subconjunctivally. The tissue may be exposed to the agent continuously or periodically. The dosage, method of application, and treatment regimen will depend on the particular agent, the type of ocular surface disorder, and the health, age and response of the subject or patient.

For the purposes of administration, the vascular permeability agents of the present invention may be formulated as pharmaceutical compositions. Such compositions comprise a vascular permeability agent and a pharmaceutically acceptable carrier and/or diluent. The agent is present in the composition in an amount which is effective to increase conjunctival vascular permeability or to treat a particular ocular surface disorder. Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats and other common additives. The compositions can also be formulated with dispersing and surface active agents, binders, or lubricants, or combinations thereof. One skilled in this art may further formulate the agent in an appropriate manner, and in accordance with accepted practices, such as those disclosed in Remington's Pharmaceutical Sciences (Gennaro, Ed., Mack Publishing Co., Easton, Pa. 1990).

In another embodiment, a method of screening a substance for treating an ocular surface disorder is provided. The foregoing descriptions as applied to treatment methods also apply to screening methods.

The present invention may be better understood by referring to the accompanying examples, which are intended for illustration purposes only and should not in any sense be construed as limiting the scope of the invention.

### Example 1

### Materials and Methods

The efficacy of C-NOD and other potential NO releasing compounds in promoting vascular permeability, and hence release of serum proteins onto the surface of the eye, was assessed in New Zealand White rabbits using two methods.

Method 1: Evans blue dye assay. Evans blue dye binds to albumin in the serum and its presence on the surface of the eye is an indicator of vascular permeability. Animals were anesthetized with 2-5% isofluorane gas administered via mask. The eyes were sterilized with 5% iodine (povidone iodine (HUMCO, Texarkana, Tex., USA) diluted to 5% in BSS (Alcon, Fort Worth, Tex., USA)) and 2-3 drops of 0.5% ophthalmic proparacaine (Alcon, Fort Worth, Tex., USA) was administered as a topical anesthetic. Right eyes (OD) received subconjunctival injections of 50 microliters of C-NOD in 100% DMSO. Left eyes (OS) received subconjunctival injections of 50 microliters of 100% DMSO. 40 mg/kg of Evans blue dye (Sigma-Aldrich, St. Louis, Mo., USA) was then injected into an ear vein. Eyes were photographed and tear samples were collected at 1, 2 and 3 days post injection. Tears were collected by insertion of 7 mm discs of Whatman 3M filter paper (Whatman, Inc., Piscataway, N.J., USA) under the lower eyelid for 2 minutes. The filter paper discs were stored in 200 microliters of a phosphate buffered saline solution containing at 5-10° C. Absorption at 620 nm was used to quantify Evans blue dye in tears.

Method 2: Total protein and serum albumin assay. Prior to subconjunctival injection, tear samples were collected from the eyes of the rabbits. Tears were collected by simultaneous insertion of two 7 mm discs of Whatman 3M filter paper (Whatman, Inc., Piscataway, N.J., USA) under the lower eyelid for 2 minutes. The filter paper discs were stored in 200 microliters of a phosphate buffered saline solution containing 0.05% Tween and 0.02% sodium azide at 5-10° C. Animals were anesthetized with 2-5% isofluorane gas administered via mask. The eyes were sterilized with 5% iodine (povidone iodine (HUMCO, Texarkana, Tex., USA) diluted to 5% in BSS (Alcon, Fort Worth, Tex., USA)) and 2-3 drops of 0.5% ophthalmic proparacaine (Alcon, Fort Worth, Texan, USA) was administered as a topical anesthetic. Right eyes (OD) received subconjunctival injections of 50 microliters of C-NOD in 100% DMSO. Left eyes (OS) received subconjunctival injections of 50 microliters of 100% DMSO. Tear samples were collected at 1 hour, then every day for up to 5 days.

Protein content of tears was assessed by two methods. First, total protein was measured using a Bio-Rad Protein Assay Kit (Bio-Rad Laboratories, Hercules, Calif., USA). Second, rabbit serum albumin was measured using an Assaypro Rabbit Albumin ELISA kit (Assaypro LLC, St. Charles, Mo., USA). Both kits were used according to the manufacturer's instructions. In some cases after 5 days, eyes were enucleated, fixed in 10% formalin, and processed for histological examination following eosin and hematoxylin staining

### Results

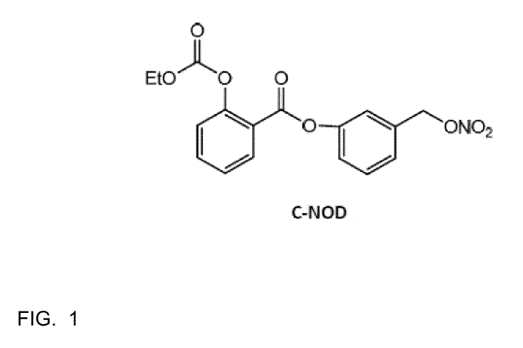

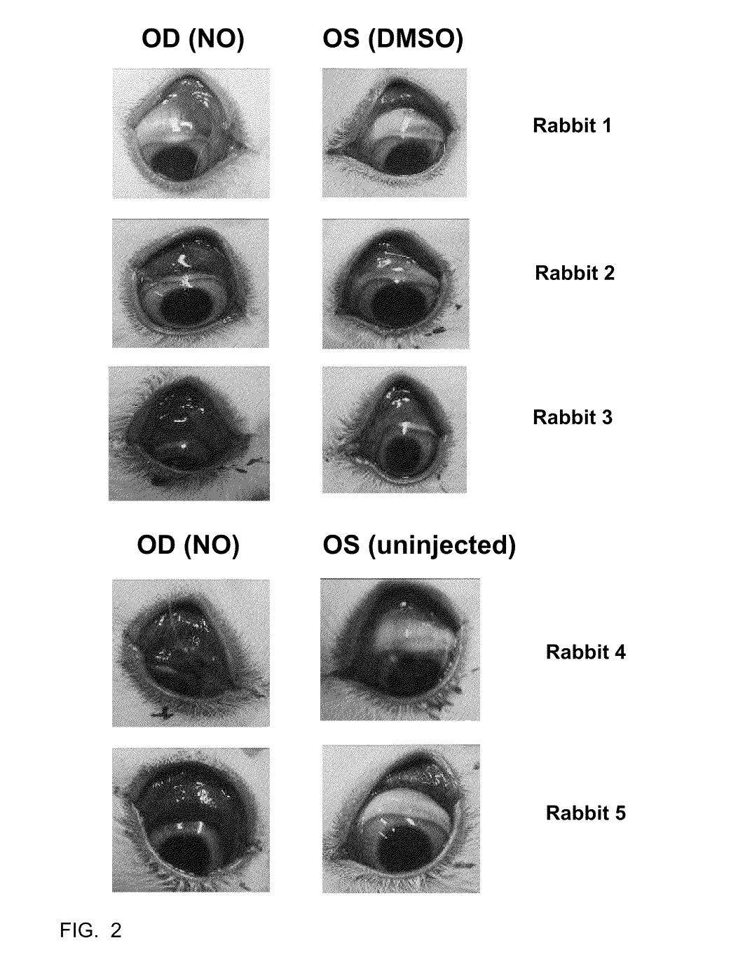

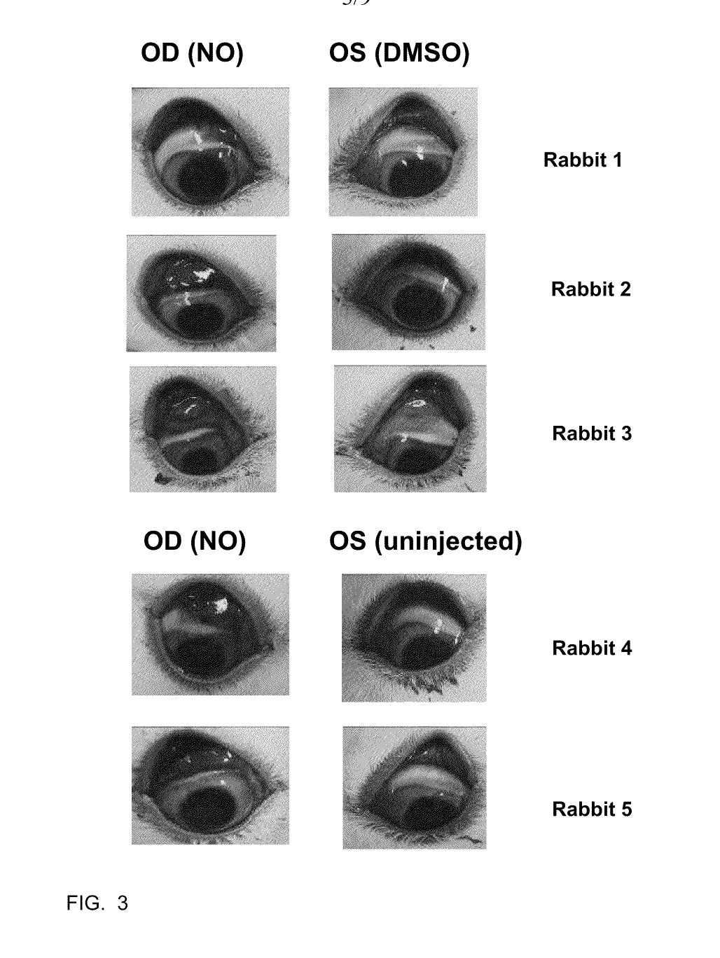

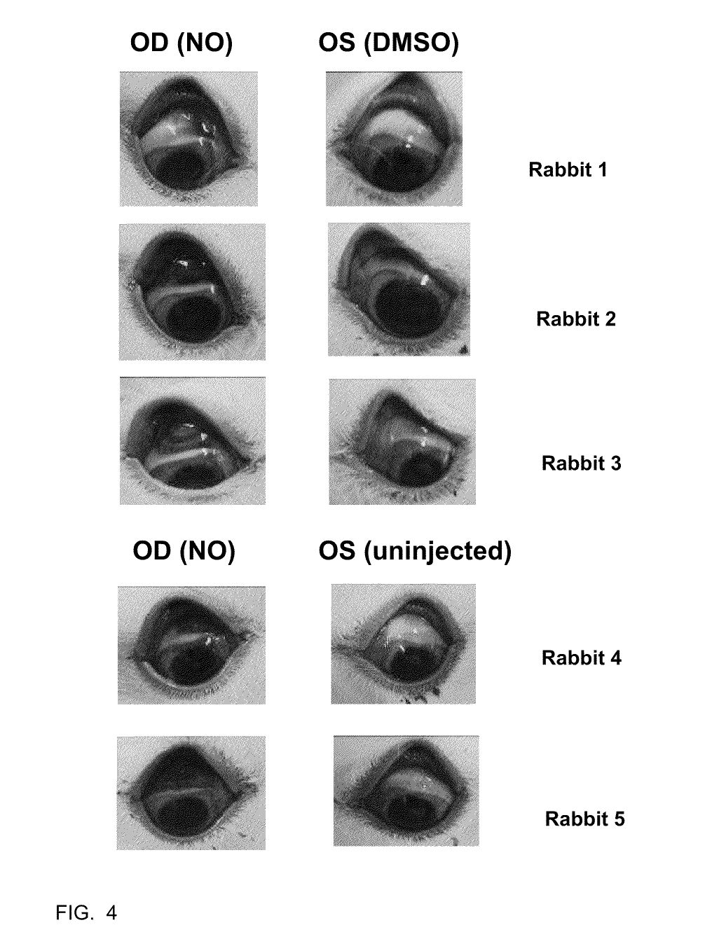

The structure of nitric oxide donor C-NOD is shown in FIG. 1. FIGS. 2-4 show the appearance of eyes of rabbits that were injected intraveinously with Evans blue dye and subconjunctivally with 66 mg/ml C-NOD at 24, 48 and 72 hours post injection. The presence of subconjunctival dark Evans blue dye in the C-NOD injected eyes that is minimal or not present at all in the vehicle injected eyes was evident.

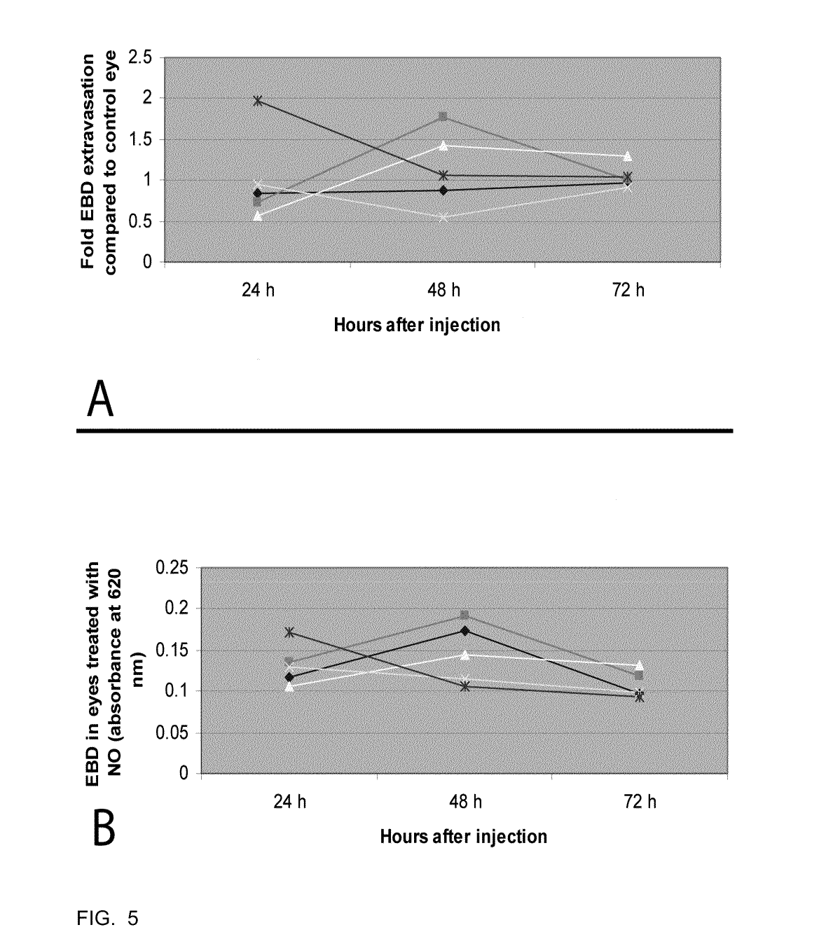

As shown in FIG. 5, in most cases, especially at 48 hours, there was more Evans blue dye present in the tears of eyes injected with C-NOD than in eyes injected with DMSO vehicle. Evans blue dye in tears was measured by absorption at 620 nm.

Rabbits were injected with 10, 25 and 50 mg/cc of C-NOD and examined clinically after 2 days. The results are outlined in Table 1.

TABLE 1
Clinical assessment of eyes injected with the indicated amounts of C-NOD

| | VEHICLE | 10 MG/CC | 25 MG/CC | 50 MG/CC |
|---|---|---|---|---|
| SUBCONJ. | TRACE TO | TRACE | TRACE | TRACE |
| HEMORRHAGE | NONE | | | |
| FOCAL | NONE | TRACE | 2+ | 4+ |
| INJECTION | | | | |
| CHEMOSIS | NONE | NONE | 2+ | 2+ |
| MUCOUS | NONE | NONE | TRACE | TRACE |

As shown in Table 1, a dose of 50 mg/cc C-NOD induces significant external inflammation of the rabbit eye compared to control saline injection. The lowest dose of C-NOD, 10 mg/cc, produces minimal post-injection conjunctival alterations, similar to a saline vehicle.

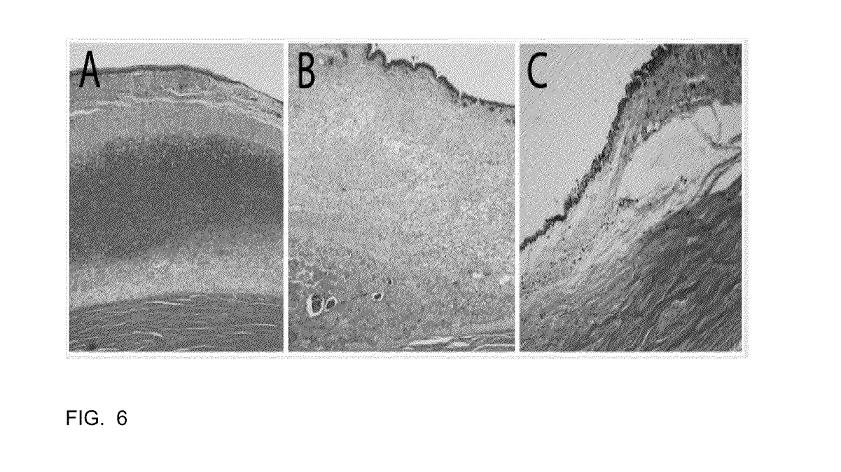

Some of the eyes that were examined at day 2 (Table 1) were subsequently enucleated on day 5 and processed for histological examination. Representative sections are shown in FIG. 6. FIG. 6 shows histology of conjunctiva 5 days after injection of C-NOD at varying concentrations. At 50 mg/cc, there was severe inflammation and conjunctival necrosis near the injection site. At 25 mg/cc, there was moderate inflammation. At 10 mg/cc there was minimal inflammation, vascular dilation, and possible mucin accumulation in cystic spaces. No other ocular histologic abnormalities noted at this concentration.

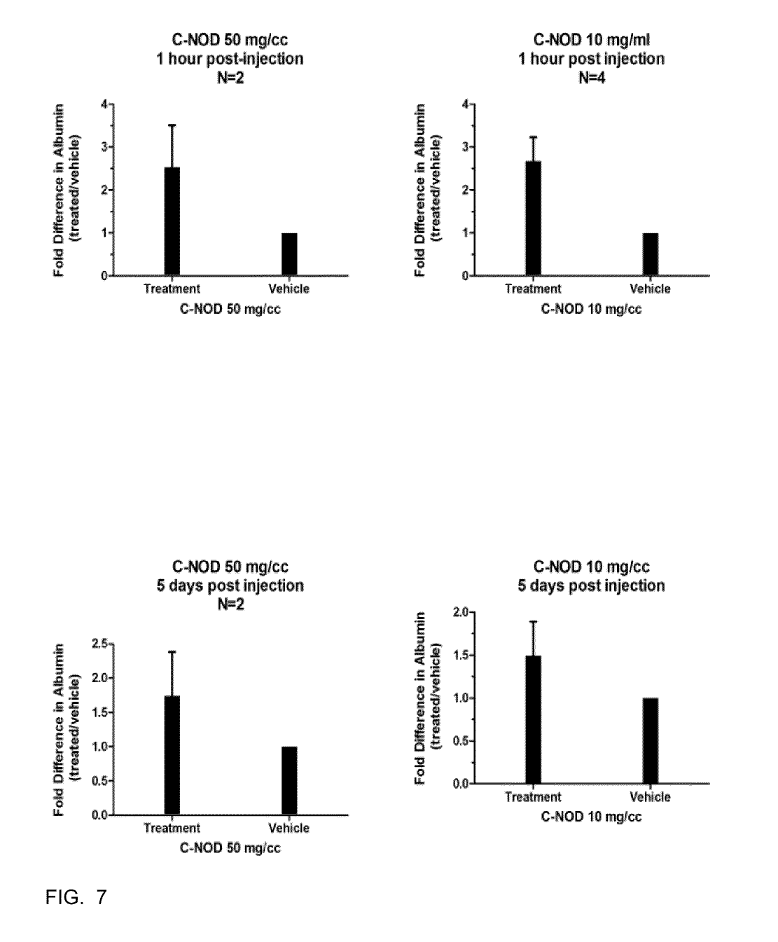

Total protein and serum albumin content of tears was measure. The results are shown in FIG. 7. For each concentration, the proportion of serum albumin in tears increased with C-NOD injection relative to vehicle injection indicating that an increased amount of tear protein was of vascular origin in C-NOD injected eyes.

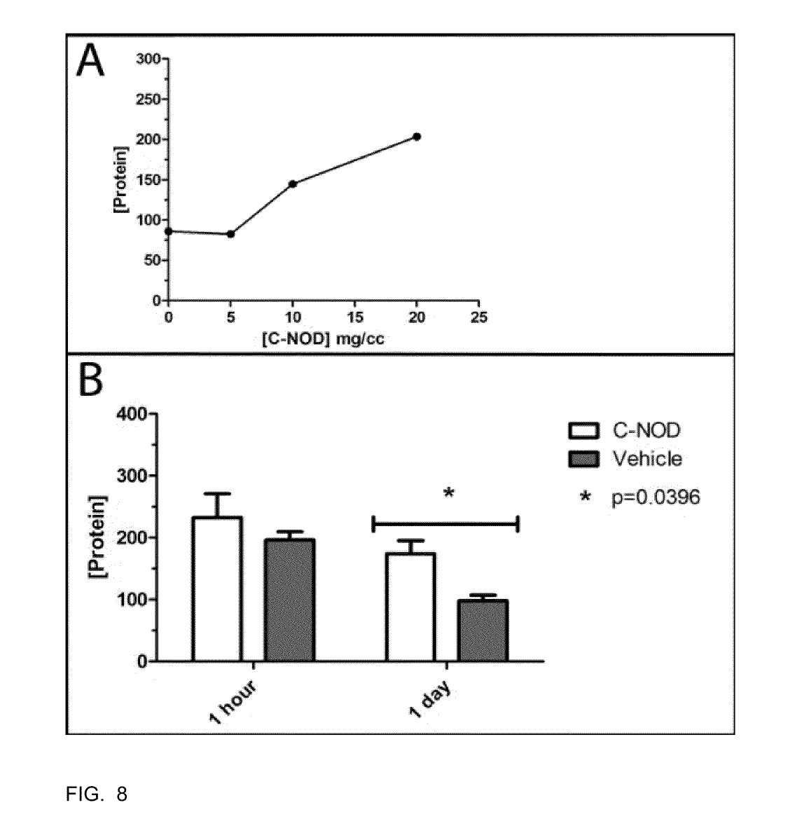

FIG. 8A shows total protein content of tears one day post-injection of C-NOD. Results are expressed as relative protein content with uninjected eyes equal to 100, and N=2 for each concentration. FIG. 8B shows total protein content in rabbit eyes following subconjunctival injection of 10 and 20 mg/cc of C-NOD at 1 day post injection. Results are expressed as relative protein content with uninjected eyes equal to 100, and N=4 for each concentration. After 1 day, injection of 10 or 20 mg/cc of C-NOD significantly increased protein content of tears (p=0.0396, two-tailed t-test). Similar results were obtained when albumin content of tears was assayed.

Initial tests on some other candidate stimulators of vascular permeability was performed. The results of clinical examination of eyes injected with the indicated compounds 1 day post-injection are shown Table 2.

TABLE 2
Clinical assessment of eyes injected with candidate compounds

| | | | Mucous |
|---|---|---|---|
| Candidate | Chemosis | Injection | discharge |
| VEGF-A (2 ng/ml) | None | None | None |
| Serotonin (100 μM) | Trace | None | None |
| Sodium nitroprusside (4 mg/ml) | None | None | None |
| Histamine (100 μM) | 3+ | Trace | Trace |
| Angiopoietin-2 (10 μg/ml) | None | None | None |

With the exception of histamine and to a lesser extent serotonin, none of the compounds induced an observable clinical response in the eyes. The testing conditions were not optimized for each candidate compound, however, which may account for a lack of observable activity.

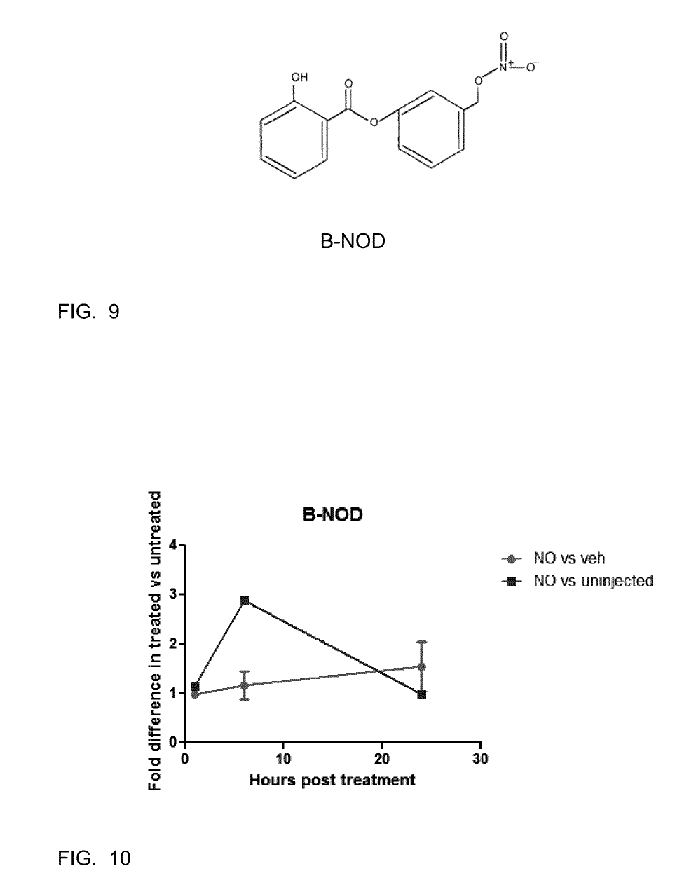

An initial test of the ability of a nitric oxide donor B-NOD (Bing, R. J., Biochem. Biophys. Res. Comm. (2000) 275, 350) to increase conjunctival vascular permeability was performed. The structure of B-NOD is shown in FIG. 9. Fifty microliters of 100 mg/ml B-NOD in DMSO was injected OD, while DMSO vehicle was injected OS. Some eyes received no injection. Vascular permeability was assessed using the Evans blue dye assay. The results are shown in FIG. 10. Six hours post-injection, tears of B-NOD injected eyes contained more Evans blue dye than did uninjected eyes. This difference disappeared by 24 hours. Throughout the experiment B-NOD injected eyes contained about the same amount of Evans blue dye as did vehicle injected eyes. The difference between B-NOD and C-NOD may be due to the shorter half-life predicted for B-NOD.

### Example 2

In this prophetic example, a patient with dry eye who has persistent symptoms and prominent rose bengal staining despite treatment with artificial tears and punctual plugs is treated with a subconjunctival injection of a sustained release NOD formulation. Topical proparacaine is instilled and a pledget containing 4% lidocaine solution is used to further anesthetize the superotemporal bulbar conjuctiva. Next, a 0.1 cc injection of subconjunctival sustained release NOD formulation is administered to the anesthetized region. Nitric oxide is released over the next 3 months causing focal conjunctival vascular dilation that is hidden by the upper lid. Serum components leak from the conjunctival vessels in the treated region for the 3 months of sustained nitric oxide release. The patient notes decreased ocular irritation. Staining of the ocular surface with rose bengal is improved compared to pre-injection. After 3 months, a repeat injection of the sustained release NOD formulation is performed.

### REFERENCES

The following publications are incorporated by reference herein:
1. Bing, R. J., et al., The pharmacology of a new nitric oxide donor: B-NOD. Biochem. Biophys. Res. Comm. (2000) 275, 350-353.
2. Fox R I, Chan R, Michelson J B, Belmont J B, Michelson P E. Beneficial effect of artificial tears made with autologous serum in patients with keratoconjunctivitis sicca. Arthritis Rheum. 1984; 27(4):459-61.
3. Jeng, B. H. and W. J. Dupps, Autologous serum 50% eyedrops in the treatment of persistent corneal epithelial defects. Cornea (2009) 28(10), 1104-1108.
4. Kojima T, Ishida R, Dogru M, Goto E, Matsumoto Y, Kaido M, Tsubota K. The effect of autologous serum eye drops in the treatment of severe dry eye disease: a prospective randomized case-control study. Am J. Ophthalmol. 2005; 139(2):242-6.
5. Lee G A, Chen S X. Autologous serum in the management of recalcitrant dry eye syndrome. Clin Experiment Ophthalmol. 2008; 36(2):119-22.
6. Leite S C, de Castro R S, Alves M, Cunha D A, Correa M E, da Silveira L A et al. Risk factors and characteristics of ocular complications, and efficacy of autologous serum tears after haematopoietic progenitor cell transplantation. Bone Marrow Transplantation, 2006; 38:223-227.
7. Noble B A, Loh R S, MacLennan S, Pesudovs K, Reynolds A, Bridges L R et al. Comparison of autologous serum eye drops with conventional therapy in a randomized controlled crossover trial for ocular surface disease. Br J. Ophthalmol. 2004; 88(5):647-52.
8. Quinto G G, Campos M, Behrens A. Autologous serum for ocular surface diseases. Arq Bras Oftalmol. 2008 November-December; 71(6 Suppl):47-54.
9. Schulze, S. D., et al., Autologous serum for treatment of corneal epithelial abrasions in diabetic patients undergoing vitrectomy. Am. J. Ophthalmol. (2006) 142, 207-211.
10. Tsubota K, Goto E, Fujita H, Ono M, Inoue H, Saito I, Shimmura S. Treatment of dry eye by autologous serum application in Sjogren's syndrome. Br J. Ophthalmol. 1999; 83(4):390-5.
11. Yamada C, King K E, Ness P M. Autologous serum eyedrops: literature review and implications for transfusion medicine specialists. Transfusion. 2008 June; 48(6):1245-55. Epub 2008 April 10. Review. PubMed PMID: 18410252.

Although the present invention has been described in connection with the preferred embodiments, it is to be understood that modifications and variations may be utilized without departing from the principles and scope of the invention, as those skilled in the art will readily understand. Accordingly, such modifications may be practiced within the scope of the invention and the following claims.

What is claimed is:

1. A method of treating an ocular surface disorder in a subject in need of such treatment, comprising increasing vascular permeability of conjunctival blood vessels of the subject.

2. The method of claim 1, wherein the increasing occurs with minimal or no external eye inflammation, or with minimal or no additional external eye inflammation over that present prior to treating.

3. The method of claim 1, wherein the increasing comprises exposing conjunctival tissue of the subject to an effective amount of a vasopermeability agent that increases conjunctival vascular permeability.

4. The method of claim 3, wherein the vasopermeability agent is a nitric oxide donor, sildenafil, histamine, serotonin, vascular endothelial growth factor (VEGF), substance P, bradykinin, platelet activating factor (PAF), TNFα, a leukotriene, a prostaglandin, an interleukin, 5-adenosine diphosphate (ADP), or a hyperosmolar agent, or a combination thereof.

5. The method of claim 4, wherein the vasopermeability agent is a nitric oxide donor.

6. The method of claim 5, wherein the nitric oxide donor is a sustained release nitric oxide donor.

7. The method of claim 6, wherein the nitric oxide donor is formulated with a carrier.

8. The method of claim 5, wherein the nitric oxide donor is 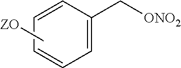
or a pharmaceutically acceptable salt thereof, wherein Z is a functional aryl or alky group.

9. The method of claim 5, wherein the nitric oxide donor is 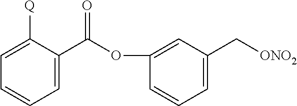
or a pharmaceutically acceptable salt thereof, wherein Q is carbamate, carbonate, urea, polymer hydroxyl, or sulfide thiourea.

10. The method of claim 5, wherein the nitric oxide donor is 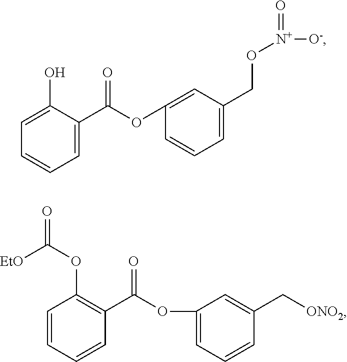
a pharmaceutically acceptable salt thereof, or a combination thereof.

11. The method of claim 3, wherein the exposing comprises applying the agent topically or subconjunctivally.

12. The method of claim 1, wherein the ocular surface disorder is dry eye disease, neurotrophic keratopathy, a non-healing ocular epithelial defect, an eye disorder due to Stevens-Johnson syndrome, an eye disorder due to graft versus host disease, Sjogren's syndrome, superior limbic keratoconjunctivitis, ocular cicatricial pemphigoid, recurrent or persistent corneal erosion, or dry eye following corneal refractive procedures (LASIK).

13. A method of screening a substance for treating an ocular surface disorder, comprising exposing conjunctival tissue to a substance, and determining if exposure to the substance increases conjunctival vascular permeability with minimal or no inflammation.

14. A nitric oxide donor of the following formula: [chemical structure]
